(19) United States
(12) Patent Application Publication
(10) Pub. No.: US 2022/0033582 A1
(43) Pub. Date: Feb. 3, 2022

(54) FLUOROPOLYETHER COMPOUND, LUBRICANT, AND MAGNETIC DISK

(71) Applicant: Moresco Corporation, Kobe-shi, Hyogo (JP)

(72) Inventor: Ryosuke SAGATA, Kobe-shi (JP)

(21) Appl. No.: 17/299,232

(22) PCT Filed: Dec. 6, 2019
(86) PCT No.: PCT/JP2019/047848

§ 371 Date: Jun. 2, 2021

(30) Foreign Application Priority Data
Dec. 6, 2018 (JP) 2018-228955

(51) Int. Cl. C08G 65/34; C10M 107/38; C10M 107/44; G11B 5/725

(52) U.S. Cl. CPC C08G 65/34; C10M 107/38; C10M 107/44; C10N 2020/04; C08G 2650/48; C10M 2213/043; C10M 2213/0606; G11B 5/725

(57) ABSTRACT

Provided is a compound which has heat resistance and decomposition resistance and with which a monomolecular film having a reduced thickness can be obtained. A fluoropolyether compound in accordance with an aspect of the present invention is represented by R¹—C₆H₄O—CH₂CH(OH)CH₂OCH₂—R²—CH₂—O—CH₂CH(OH)CH₂—OC₆H₄—R¹ where R¹ is a hydrogen atom, an alkoxy group having 1 to 4 carbon atoms, an amino group, or an amide group, R² is —CF₂O(CF₂CF₂O)ₓCF₂—, and x is a real number of 1 to 35.

### TECHNICAL FIELD

The present invention relates to a fluoropolyether compound, a lubricant, and a magnetic disk.

### BACKGROUND ART

Many of the existing magnetic disks are constituted by: a recording layer disposed on a substrate; a protective layer disposed on the recording layer in order to protect information recorded on the recording layer; and a lubricant layer disposed on the protective layer.

Relating to such magnetic disks, for example, technologies disclosed in Patent Literatures 1 through 3 are known. Patent Literatures 1 through 3 respectively have the objective of providing a highly heat resistant lubricant, the objective of providing a lubricant that is excellent in LUL durability and alumina resistance, and the objective of providing a lubricant that has good fluidity and adsorption and is thermally stable. For these objectives, fluoropolyether compounds each having a specific structure are used in respective Patent Literatures 1 through 3.

### CITATION LIST

### Patent Literature

[Patent Literature 1]
International Publication No. WO 2015/087615 (Publication Date: Jun. 18, 2015)

[Patent Literature 2]
Japanese Patent Application Publication Tokukai No. 2009-266360 (Publication date: Nov. 12, 2009)

[Patent Literature 3]
Japanese Patent Application Publication Tokukai No. 2010-143855 (Publication date: Jul. 1, 2010)

### SUMMARY OF INVENTION

### Technical Problem

In heat-assisted magnetic recording (HAMR), which is a next-generation magnetic recording technique, local heating can be performed using laser. In a case where a lubricant is decomposed in such a situation, the decomposition may cause malfunction.

Further, with the increasing recording density of magnetic disks in recent years, the distance between a magnetic head and the surface of a magnetic disk has decreased to the order of ten nanometers to read data from very small recording magnetic domains. This has led to a demand for a lubricant layer having an even thinner film thickness.

Ideally, therefore, a lubricant for HAMR is required to undergo no heat decomposition even at high temperature and to allow reducing the distance (HMS) between a head and a magnetic layer. However, no lubricant that meets these requirements has been developed, and the conventional technologies as described above have room for improvement.

An objective of an aspect of the present invention is to provide a compound which has heat resistance and decomposition resistance and with which a monomolecular film having a reduced thickness can be obtained.

### Solution to Problem

The inventor of the present invention conducted diligent research to attain the above objectives, and synthesized a fluoropolyether compound having a specific structure different from those in Patent Literatures 1 through 3. The inventor found that the fluoropolyether compound has heat resistance and decomposition resistance, and a monomolecular film obtained with the fluoropolyether compound has a reduced thickness. On the basis of this finding, the inventor completed the present invention. Specifically, the present invention encompasses the following arrangements.

[1] A fluoropolyether compound represented by Formula (1) below:

R¹—C₆H₄O—CH₂CH(OH)CH₂OCH₂—R²—CH₂—O—CH₂CH(OH)CH₂—OC₆H₄—R¹ (1)

where R¹ is a hydrogen atom, an alkoxy group having 1 to 4 carbon atoms, an amino group, or an amide group, R² is —CF₂O(CF₂CF₂O)ₓCF₂—, and x is a real number of 1 to 35.
[2] A lubricant including a fluoropolyether compound recited in [1].
[3] A magnetic disk including: a recording layer; a protective layer disposed on the recording layer; and a lubricant layer disposed on the protective layer, the lubricant layer including a lubricant recited in [2].

### Advantageous Effects of Invention

According to an aspect of the present invention, it is possible to provide a compound which has heat resistance and decomposition resistance and with which a monomolecular film having a reduced thickness can be obtained.

### BRIEF DESCRIPTION OF DRAWINGS

FIG. 1 shows cross-sectional views illustrating structures of magnetic disks in accordance with embodiments of the present invention.

### DESCRIPTION OF EMBODIMENTS

The following description will discuss embodiments of the present invention in detail. Note, however, that the present invention is not limited to the following embodiments, but can be altered within this disclosure. The present invention also encompasses, in its technical scope, any embodiment derived by combining technical means disclosed in differing embodiments. Note that the expression “A to B”, representing a numerical range, herein means “not less than A and not more than B” unless otherwise specified in this specification.

[1. Fluoropolyether Compound]

A fluoropolyether compound in accordance with an embodiment of the present invention is represented by Formula (1) below:

R¹—C₆H₄O—CH₂CH(OH)CH₂OCH₂—R²—CH₂—O—CH₂CH(OH)CH₂—OC₆H₄—R¹ (1)

where R¹ is a hydrogen atom, an alkoxy group having 1 to 4 carbon atoms, an amino group, or an amide group, R² is —CF₂O(CF₂CF₂O)ₓCF₂—, and x is a real number of 1 to 35.

As a compound used in conventional lubricants, the following compounds are known: a compound having CF₂CF₂CF₂O (also referred to as “Demnum skeleton” in this specification) as a repeating unit in R², and a compound having CF₂CF₂CF₂CF₂O (also referred to as “C4 skeleton” in this specification) as a repeating unit in R², as described in Patent Literature 1. Other known compounds include a compound having a skeleton (also referred to as “Fomblin skeleton” in this specification) in which CF₂CF₂O and CF₂O are randomly repeated, as described in Patent Literatures 2 and 3. The Demnum skeleton, for example, has a repeating unit formed of an odd number of carbon atoms and thus tends to have an arched protruding shape. The Fomblin skeleton has a random, and thus spiral, structure. Accordingly, the Fomblin skeleton also tends to have an arched protruding shape.

In contrast, a fluoropolyether compound in accordance with an embodiment of the present invention has a repeating unit (also referred to as “C2 skeleton” in this specification) represented by CF₂CF₂O in R². The C2 skeleton has a structure in which two carbon atoms and an ether linkage are repeated. Accordingly, the fluoropolyether compound has a substantially linear structure. That is, the fluoropolyether compound has a molecular chain which is more flat than those of the conventional compounds. As such, in a case where the fluoropolyether compound is used in a lubricant layer of a magnetic disk, it is possible to reduce the film thickness per molecule, i.e., the monomolecular film thickness, in comparison to the conventional lubricants. This enables a reduction in HMS.

Further, since the C2 skeleton described above does not have a C1 unit (CF₂O), which has a short distance between ethers and is easily decomposed when heated, the C2 skeleton is less easily decomposed by heat at high temperature in comparison to the Fomblin skeleton and the like.

CF₂O, which is a main chain of a fluoropolyether compound, is considered to be easily decomposed by a Lewis acid such as alumina (Al₂O₃) contained in a slider of a magnetic head. However, a fluoropolyether compound in accordance with an embodiment of the present invention includes an aromatic group (—C₆H₄O—), and this portion (site) is coordinated to a Lewis acid to inactivate the Lewis acid. This inhibits a catalytic decomposition activity, and thus makes it difficult for the main chain to be decomposed.

Examples of the alkoxy group having 1 to 4 carbon atoms include a methoxy group, an ethoxy group, a propoxy group, and a butoxy group. Examples of the amino group include an amino group, a methyl amino group, a dimethylamino group, an ethylamino group, and a diethylamino group. Examples of the amide group include an acetamide group and a propionamide group.

x is more preferably a real number of 1 to 25, even more preferably a real number of 1 to 15, particularly preferably a real number of 5 to 12. In particular, when x is a real number of 5 to 12, the molecular chain of the fluoropolyether compound becomes more flat, making it possible to form a thin film of a lubricant that contains the fluoropolyether compound.

[2. Method of Producing Fluoropolyether Compound]

The fluoropolyether compound can be produced, for example, by allowing a linear fluoropolyether (a) having hydroxyl groups at both terminals thereof and a phenoxy compound having an epoxy group to react with each other.

The linear fluoropolyether (a) having hydroxyl groups at both terminals thereof may be, for example, a compound represented by HOCH₂CF₂O(CF₂CF₂O)ₓCF₂CH₂OH. The number average molecular weight of this fluoropolyether is preferably 500 to 4000, more preferably 800 to 1500. Note here that the number average molecular weight is a value determined by ¹⁹F-NMR using JNM-ECX400 available from JEOL Ltd. In the NMR determination, a sample is used as it is without being diluted with a solvent. A known peak that is part of the skeleton structure of the fluoropolyether can be used as a reference for the chemical shift. x is a real number of 1 to 35, more preferably a real number of 5 to 12. When x is a real number of 5 to 12, the molecular chain of the fluoropolyether becomes more flat. Thus, it is preferable that x be a real number of 5 to 12.

The fluoropolyether (a) is a compound having a molecular weight distribution, and preferably has a molecular weight distribution (PD) represented by weight average molecular weight/number average molecular weight of 1.0 to 1.5, more preferably 1.0 to 1.3, even more preferably 1.0 to 1.1. Note that the molecular weight distribution is a property value obtained with use of HPLC-8220GPC available from Tosoh Corporation, a column (PLgel Mixed E) available from Polymer Laboratories, a HCFC-based substitute for CTCs as an eluent, and a non-functional perfluoropolyether as a standard substance.

Examples of the phenoxy compound having an epoxy group include a compound represented by Formula (A) below.

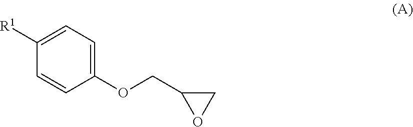

where R¹ is an alkoxy group having 1 to 4 carbon atoms, an amino group, or an amide group, and exemplified by those described above in [1. Fluoropolyether compound].

Specific examples of the compound (A) include glycidyl 4-methoxyphenyl ether, glycidyl 4-ethoxyphenyl ether, glycidyl 4-propoxyphenyl ether, glycidyl 4-butoxyphenyl ether, glycidyl 4-aminophenyl ether, glycidyl 4-methylaminophenyl ether, glycidyl 4-dimethylaminophenyl ether, glycidyl 4-ethylaminophenyl ether, glycidyl 4-diethylaminophenyl ether, glycidyl 4-acetamidephenyl ether, and glycidyl 4-propionamidephenyl ether.

A fluoropolyether compound in accordance with an embodiment of the present invention can be synthesized, specifically, by the following method. First, a linear fluoropolyether (a) having hydroxyl groups at both terminals thereof and a phenoxy compound (A) having an epoxy group are allowed to react with each other in the presence of a catalyst. The reaction temperature is preferably 20° C. to 90° C., more preferably 60° C. to 80° C. The reaction time is preferably 5 hours to 20 hours, more preferably 10 hours to 15 hours. It is preferable that the phenoxy compound (A) be used in an amount of 1.0 to 2.0 equivalents of the linear fluoropolyether (a), and the catalyst be used in an amount of 0.05 to 0.1 equivalents of the linear fluoropolyether (a). As the catalyst, an alkaline compound such as sodium t-butoxide and potassium t-butoxide can be used. The reaction may be carried out in a solvent. Examples of the solvent include t-butanol, toluene, and xylene. Then, a product obtained from the reaction is, for example, washed with water and dehydrated. Thus, the fluoropolyether compound represented by Formula (1) above is obtained.

[3. Lubricant]

A lubricant in accordance with an embodiment of the present invention contains a fluoropolyether compound in accordance with an embodiment of the present invention. With regard to a lubricant, a fluoropolyether compound in accordance with an embodiment of the present invention may be used alone as a lubricant. Alternatively, a fluoropolyether compound in accordance with an embodiment of the present invention and some other component mixed at a certain ratio may be used as a lubricant, provided that the performance of the fluoropolyether compound is not impaired.

Examples of the above-described other component include: known lubricants for magnetic disks such as Fomblin (registered trademark) Zdol (available from Solvay Solexis), Ztetraol (available from Solvay Solexis), Demnum (registered trademark) (available from Daikin Industries, Ltd.), and Krytox (registered trademark) (available from DuPont); PHOSFAROL A20H (MORESCO PHOSFAROL A20H) (available from MORESCO Corporation); and MORESCO PHOSFAROL D-4OH (available from MORESCO Corporation).

The lubricant can be used as a lubricant for storage media, in order to improve the sliding properties of magnetic disks. The lubricant can also be used as a lubricant for storage media in other recording devices that involve sliding between a recording medium (e.g., a magnetic tape) other than magnetic disks, and a head. The lubricant can also be used as a lubricant for other devices having a part involving sliding, not confined to the recording devices.

[4. Magnetic Disk]

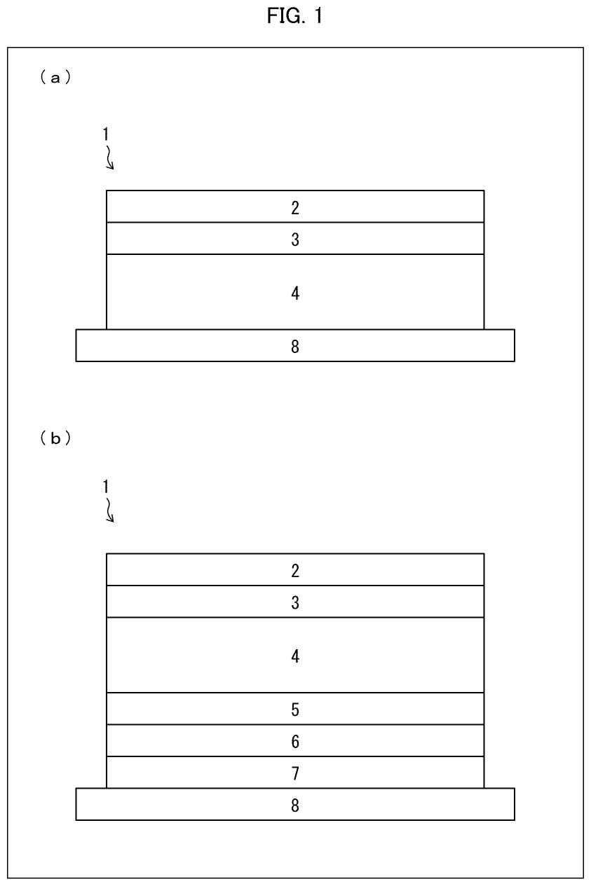

A magnetic disk 1 in accordance with an embodiment of the present invention includes, as illustrated in (a) of FIG. 1, a recording layer 4, a protective film layer (protective layer) 3, and a lubricant layer 2, which are disposed on a non-magnetic substrate 8. The lubricant layer 2 contains the foregoing lubricant.

In another embodiment, a magnetic disk can include, like a magnetic disk 1 illustrated in (b) of FIG. 1, a lower layer 5 that underlies the recording layer 4, one or more soft magnetic lower layers 6 that underlie the lower layer 5, and an adhesive layer 7 that underlies the one or more soft magnetic lower layers 6. In one embodiment, all these layers can be formed on the non-magnetic substrate 8.

Each of the layers of the magnetic disk 1 other than the lubricant layer 2 can contain a material that is known in this technical field to be suitable for a corresponding layer of a magnetic disk. Examples of the material of the recording layer 4 include: an alloy of an element (e.g., iron, cobalt, and nickel) from which a ferromagnetic material can be formed and chromium, platinum, tantalum or the like; and an oxide of the alloy. Examples of the material of the protective layer 3 include carbon, Si₃N₄, SiC, and SiO₂. Examples of the material of the non-magnetic substrate 8 include an aluminum alloy, glass, and polycarbonate.

[5. Method of Producing Magnetic Disk]

A method of producing a magnetic disk in accordance with an aspect of the present invention includes a step of forming a lubricant layer by placing a lubricant in accordance with an embodiment of the present invention on the exposed surface of a protective layer of a stack of a recording layer and the protective layer.

There is no particular limitation on a method of forming a lubricant layer by placing the lubricant on the exposed surface of a protective layer of a stack of a recording layer and the protective layer. It is preferable that a lubricant be placed on the exposed surface of a protective layer by the following method: the lubricant is diluted with a solvent and then placed on the exposed surface. Examples of the solvent include: PF-5060, PF-5080, HFE-7100, and HFE-7200 available from 3M; and Vertrel-XF (registered trademark) available from DuPont. The lubricant diluted with a solvent has a concentration of preferably 0.001 wt % to 1 wt %, more preferably 0.005 wt % to 0.5 wt %, even more preferably 0.01 wt % to 0.1 wt %. When the concentration of the lubricant diluted with a solvent is 0.01 wt % to 0.1 wt %, the viscosity of the lubricant is low enough to easily control the thickness of the lubricant layer.

The following arrangement may be employed: the recording layer and the protective layer are formed in this order; the lubricant is placed on the exposed surface of the protective layer; and then ultraviolet irradiation or heat treatment is carried out.

Carrying out ultraviolet irradiation or heat irradiation forms stronger bonds between the lubricant layer and the exposed surface of the protective layer and, in turn, prevents the lubricant from evaporating from heat. When carrying out ultraviolet irradiation, it is preferable to use an ultraviolet ray having a wavelength of 185 nm or 254 nm as the dominant wavelength, in order to activate the exposed surface without affecting deep areas of the lubricant layer and the protective layer. The temperature of the heat treatment is preferably 60° C. to 170° C., more preferably 80° C. to 170° C., even more preferably 80° C. to 150° C.

### EXAMPLES

The following description will more specifically discuss the present invention based on Examples; however, the present invention is not limited to the following Examples.

[Evaluation of Heat Resistance]

Lubricants to be described later were heated with use of a thermal analysis apparatus (TG/DTA) at 2° C./min in a nitrogen atmosphere. The heat resistance of the lubricants was evaluated on the basis of a temperature at which the lubricants had been reduced by 10%.

[Evaluation of Decomposition Resistance with Respect to Aluminum Oxide]

20% by weight of Al₂O₃ was added to each of the lubricants to be described later, and the resultant mixture was vigorously shaken and then further mixed well ultrasonically to prepare a sample for evaluation of decomposition resistance. A thermal analysis apparatus (TG/DTA) was used to calculate a weight reduction rate (B) of the lubricants after heating at 250° C. for 100 minutes. Further, the lubricants were subjected to a similar thermal analysis in which the lubricants were used as they were in an amount of 20 mg each without addition of Al₂O₃, to calculate a weight reduction rate (C) of the lubricates. The decomposition resistance of the lubricants was evaluated on the basis of a difference (B−C) between B and C.

[Evaluation of Monomolecular Film Thickness]

The lubricants to be described later were each dissolved in Vertrel-XF available from DuPont. The concentration of the lubricants in this solution was 0.05% by weight. A portion (approximately ¼) of a magnetic disk of 2.5 inches in diameter was immersed in the solution and then pulled out at a speed of 4 mm/s to prepare a disk including a lubricant layer consisting of a portion (coated part) coated with the lubricants and a portion (non-coated part) not coated with the lubricants. The coated part had an average thickness of 20 Å.

Immediately after the disk was prepared, the disk was set on an ellipsometer, and then a change in film thickness near a boundary between the coated part and the non-coated part was measured every certain period of time under a temperature condition of 50° C. A terrace part was formed, and the thickness of the terrace part was treated as the thickness of a monomolecular film of the lubricants.

### Example 1

### Synthesis of CH₃O—C₆H₄O—CH₂CH(OH)CH₂OCH₂—CF₂O(CF₂CF₂O)ₓCF₂CH₂—O—CH₂CH(OH)CH₂—OC₆H₄—O—CH₃ (Compound 1)

In an argon atmosphere, a mixture of t-butyl alcohol (21 g), 50 g of a fluoropolyether represented by HO—CH₂CF₂O(CF₂CF₂O)ₓCF₂—CH₂—OH (number average molecular weight: 999, molecular weight distribution: 1.46), potassium t-butoxide (1.1 g), and 4-methoxyglycidylphenyl ether (20 g) was stirred at 70° C. for 16 hours. Then, the obtained mixture was washed with water, dehydrated, and purified by distillation, thereby giving 42 g of Compound 1.

Compound 1 was a yellow transparent liquid, and the density of Compound 1 was 1.72 g/cm³ at 20° C. Compound 1 was identified by NMR. The results of the NMR are as follows. ¹⁹F-NMR (solvent: none, standard substance: OCF₂CF₂O in the product [−89.1 ppm])

δ=−89.1 ppm

[14F, —OCF₂CF₂O—],

δ=−78.0 ppm

[4F, —OCF₂CH₂OCH₂CH(OH)CH₂—O—C₆H₄—OCH₃],

x=7.1

1H-NMR (solvent: none, standard substance: D₂O)

δ=3.2 ppm to 3.8 ppm

[22H, H₃CO—C₆H₄O—CH₂CH(OH)CH₂O—CH₂CF₂CF₂O(CF₂CF₂CF₂O)_{z}CF₂CF₂CH₂—OCH₂CH(OH)CH₂—OC₆H₄—OCH₃]

δ=6.1 ppm, 6.7 ppm

[8H, —OCF₂CF₂CH₂OCH₂CH(OH)CH₂—C₆H₄—OCH₃]

The obtained compound 1 was used as a lubricant of Example 1.

### Comparative Example 1

As Comparative Example 1, a lubricant 2 having aromatic groups at both terminals of a perfluoropolyether and having a Demnum skeleton as shown below was used.

CH₃O—C₆H₄O—CH₂CH(OH)CH₂OCH₂—CF₂CF₂O(CF₂CF₂CF₂O)_{z}CF₂CF₂—CH₂—O—CH₂CH(OH)CH₂OC₆H₄O—CH₃

Note that z is 4.2, and the molecular weight distribution is 1.41.

### Comparative Example 2

As Comparative Example 2, a lubricant 3 having aromatic groups at both terminals of a perfluoropolyether and having a C4 skeleton as shown below was used.

CH₃O—C₆H₄O—CH₂CH(OH)CH₂OCH₂—CF₂CF₂CF₂O(CF₂CF₂CF₂CF₂O)ₙCF₂CF₂CF₂—CH₂—O—CH₂CH(OH)CH₂OC₆H₄O—CH₃

Note that n is 3.0, and the molecular weight distribution is 1.58.

### Comparative Example 3

As Comparative Example 3, a lubricant 4 having aromatic groups at both terminals of a perfluoropolyether and having a Fomblin skeleton was used.

CH₃O—C₆H₄O—CH₂CH(OH)CH₂OCH₂—CF₂O(CF₂CF₂O)ᵥ(CF₂O)_{w}CF₂—CH₂—O—CH₂CH(OH)CH₂—OC₆H₄O—CH₃

Note that v is 5.4, w is 5.2, and the molecular weight distribution is 1.50.

[Results]

Evaluation results are shown in Table 1 below.

TABLE 1

| | | Decomposition | |
|---|---|---|---|
| | | resistance with | |
| | | respect to | Monomolecular |
| | Heat resistance | aluminum oxide | film thickness |
| Sample | (° C.) | (%) | (Å) |
| Example 1 | 284 | 38 | 16 |
| Comparative | 300 | 21 | 19 |
| Example 1 | | | |
| Comparative | 302 | 22 | 18 |
| Example 2 | | | |
| Comparative | 252 | 37 | 20 |
| Example 3 | | | |

It is understood from Table 1 that a monomolecular film of the lubricant of Example 1 was further reduced in thickness in comparison to the lubricants of Comparative Examples 1 through 3, while having heat resistance and decomposition resistance similar to those of the lubricants of Comparative Examples 1 through 3. That is, it was confirmed that a compound in accordance with an embodiment of the present invention is suitable for use in preparation of a lubricant and a magnetic disk, because the compound has heat resistance and decomposition resistance, and a monomolecular film obtained with the compound has a reduced thickness.

### INDUSTRIAL APPLICABILITY

A fluoropolyether compound in accordance with an aspect of the present invention is suitable for use as a lubricant for a magnetic disk.

### REFERENCE SIGNS LIST

1 magnetic disk
2 lubricant layer
3 protective film layer (protective layer)
4 recording layer
5 lower layer
6 soft magnetic lower layer
7 adhesive layer
8 non-magnetic substrate

1. A fluoropolyether compound represented by Formula (1) below:
R¹—C₆H₄O—CH₂CH(OH)CH₂OCH₂—R²—CH₂—O—CH₂CH(OH)CH₂—OC₆H₄—R¹ (1)
where R¹ is a hydrogen atom, an alkoxy group having 1 to 4 carbon atoms, an amino group, or an amide group, R² is —CF₂O(CF₂CF₂O)ₓCF₂—, and x is a real number of 1 to 35.

2. A lubricant comprising a fluoropolyether compound recited in claim 1.

3. A magnetic disk comprising: a recording layer; a protective layer disposed on the recording layer; and a lubricant layer disposed on the protective layer,
the lubricant layer comprising a lubricant recited in claim 2.
